(19) United States
(12) Patent Application Publication
(10) Pub. No.: US 2010/0044560 A1
(43) Pub. Date: Feb. 25, 2010

(54) Method and Apparatus for Pyrolysis-Induced Cleavage in Peptides and Proteins

(76) Inventors: Franco Basile, Ft. Collins, CO (US); Shaofeng Shang, Exton, PA (US)

Correspondence Address: DAVIS, BROWN, KOEHN, SHORS & ROBERTS, P.C.;THE DAVIS BROWN TOWER, 215 10TH STREET SUITE 1300, DES MOINES, IA, 50309, US

(21) Appl. No.: 12/307,538

(22) PCT Filed: Jul. 5, 2007
(86) PCT No.: PCT/US07/15444

§ 371 Date: Oct. 12, 2009

Related U.S. Application Data
(60) Provisional application No. 60/818,858, filed on Jul. 6, 2006.

(51) Int. Cl. B01D 59/44; C07K 1/14; H01J 49/00

(52) U.S. Cl. 250/282; 530/344; 250/281

(57) ABSTRACT

A method and apparatus for conducting the rapid pyrolysis of peptides, proteins, polymers, and biological materials. The method can be carried out at atmospheric pressures and takes only about 5 to 30 seconds. The samples are cleaved at the C-terminus of aspartic acid. The apparatus employs a probe on which the sample is heated and digested components analyzed.

The United States Government has rights in this invention under National Institutes of Health—National Center for Research Resource (R15-RR020354-01A1) and United States Department of Agriculture (USDA Grant #448800).

### BACKGROUND OF THE INVENTION

This application claims priority to U.S. Patent Application Ser. No. 60/818,858, filed Jul. 6, 2006.

The invention relates generally to pyrolysis-induced cleavage of peptides and proteins and, more specifically, to

A simple and site-specific nonenzymatic method based on pyrolysis has been developed to cleave peptides and proteins. Pyrolytic cleavage was found to be specific and rapid as it induced a cleavage at the C-terminal side of aspartic acid in the temperature range of 220-250° C. in 10 s. Electrospray ionization (ESI) mass spectrometry (MS) and tandem-MS (MS/MS) were used to characterize and identify pyrolysis cleavage products, confirming that sequence information is conserved after the pyrolysis process in both peptides and protein tested. This suggests that pyrolysis-induced cleavage at aspartyl residues can be used as a rapid protein digestion procedure for the generation of sequence-specific protein biomarkers.

Protein digestion along with either peptide mass mapping or sequence-specific mass spectra forms part of a powerful bottom-up method for protein identification and characterization. This approach has been made possible by advances in both mass analyzer designs and the advent of new ionization techniques like matrix-assisted laser desorption/ionization (MALDI) and electrospray ionization (ESI). Digestion of proteins into peptides is usually carried out by enzymatic action, commonly tryptic, along with chemical methods like CNBr cleavage at methionine and oxidative chemical cleavage at tyrosine and trytophan. Even though these methods provide the required site-specificity for successful database search and protein identification, they depend on relatively slow enzymatic activity or require time-consuming or labor intensive procedures. Moreover, tryptic-based approaches may not be particularly suited for proteins lacking arginine and/or lysine amino acids or non-soluble proteins. In addition, for applications requiring automated and field-portable instrumentation and using proteomic-based analyses, approaches using enzymatic digestion may add to the complexity and cost of the final field-portable device. It is with this focus on automation and miniaturization of the sample preparation step for bottom-up proteomic analyses for microorganism detection (i.e., biodetection) that our laboratory is developing rapid reagentless approaches for site-specific cleavage of peptides and proteins based on pyrolysis, electrochemical oxidation, and microwave-heated mild acid hydrolysis.

Pyrolysis has been widely used as a sample preparation step in the analysis of low molecular weight volatile products by mass spectrometry. More recently, however, the focus has been shifted to the analysis of nonvolatile pyrolysis products of biological and synthetic polymers by MALDI-MS.

Besides offering the ability to analyze the intact synthetic polymer molecules, ESI and MALDI allow the analysis of the non-volatile pyrolysis products of these compounds. MALDI-MS is particularly well suited for the analysis of high molecular weight mixtures and complex synthetic polymer compounds due to the predominant singly charged nature of the signals generated. The use of MALDI-MS to study non-volatile pyrolysis products was first demonstrated with the analysis of pyrolytic products of segmented polyurethane. This study identified several series of oligomeric non-volatile products over the mass range˜800-10,000 Da, including linear and cyclic polyester oligomers. MALDI-MS was also employed to study low-temperature pyrolysis products from poly(ethylene glycol). This last study found that the dominant oligomeric products had hydroxyl and ethyl ether end groups, while at higher temperatures, methyl ether and vinyl ether end groups became more abundant in the pyrolyzates. Other studies have also used MALDI-MS for the study of thermal oxidative degradation of nylon-6 and the thermal degradation of aromatic poly(carbonate) polymers in the temperature range of 300-700° C. Pyrolysis was also combined with MALDIMS to study the non-volatile pyrolysis products of poly-amino acids and a small protein pyrolyzed in a nitrogen atmosphere and at temperatures ranging from 245 to 285° C. In this last study, the pyrolysis products were extracted and analyzed by MALDI-MS and it was hypothesized that the amino acid chains undergo dehydration through the formation of cyclic oligopeptides. In addition, the use of ESI-MS for the analysis of nonvolatile pyrolysis products was demonstrated with the pyrolysis of dimethylamphetamine and the analysis of thermal decomposition of three common pharmaceuticals: acetaminophen, indomethacin, and mefenamic acid. In all these studies, however, sample preparation was required and involved dissolving and extracting the non-volatile residues with appropriate solvents (ESI) or mixing with matrices (MALDI). This sample pre-processing step increases analysis time and could possibly affect the analysis by introducing a sampling bias and consequently not detecting important products. The introduction of ambient MS techniques has brought a new dimension in mass spectrometric measurements as they allow the analysis of samples in their native environment. To date, a number of ambient ionization methods for MS analysis have been introduced, but most notably are direct analysis in real-time (DART) and desorption electrospray ionization (DESI). Of interest to this investigation is the ability of DESI to ionize compounds from surfaces with a mechanism similar to conventional ESI and its applicability to analytes of a wide range of molecular weights. These analytes include, but are not limited to, pharmaceuticals and controlled substances, peptides and proteins explosives, clinical samples, intact tissues, synthetic polymers and bacteria. DESI is a rapid desorption/ionization source for MS and requires little to no sample preparation. DESI is carried out by directing aerosolized and electrosprayed charged droplets and ions of solvent onto the surface to be analyzed. The charged droplets impact on the surface and “pick up” available soluble molecules. These charged droplets subsequently “bounce” at a lower angle towards the MS inlet and yield gaseous ions of the compound in an analogous mechanism to that in ESI. Hence, DESI yields mass spectra similar to those obtained by ESI which are characterized by multiply charged ions and are amenable for tandem mass analysis (MS/MS). However, it is reasonable to assume that the nature and polarity of the DESI solvent can be varied to affect sampling of pyrolysis products during the surface pick up step of the DESI process.

### SUMMARY OF THE INVENTION

The present invention consists of heating a protein sample defined as a pure protein, a mixture of proteins, whole microorganisms or intact tissue, to pyrolytic temperatures in a short period of time. Preferably, the sample is heated to between about 180° C. and about 250° C., and most preferably to between about 210° C. and 230° C., in a period of between about 5 seconds and about 30 seconds, and most preferably in about 10 seconds. This can be carried out under atmospheric conditions.

The present invention in preferred embodiments consists of the use of pyrolysis as a sample preparation technique by applying pyrolysis as a site-specific peptide and protein cleavage method. This methodology is found to specifically induce hydrolysis at the C-terminus of the aspartic acid residue in a polypeptide chain in less than 10 seconds. Peptides containing aspartic acid were tested along with the protein lysozyme. Tandem MS (MS/MS) results confirm cleavage at the C-terminus of aspartic acid.

An alternative embodiment of the present invention consists of an on-probe pyrolyzer interfaced to a desorption electrospray ionization (DESI) source as an in situ and rapid pyrolysis technique to investigate non-volatile pyrolytic residues by MS and MS/MS analyses. The technique is useful in sample analysis, including the analysis of biological samples and synthetic polymers.

The purpose of this invention is the rapid and non-enzymatic of peptides and proteins at specific amino acid positions with rapid heating. The invention can be used in proteomic applications to where the purpose is to identify the original protein. The invention being described here achieves the level of site-specificity, is very rapid and uses no enzymes.

The invention has advantages over the enzymatic approach in that it is rapid and inexpensive. The invention performs the digestion in 10 seconds as compared to the several hours to overnight incubation required for the enzyme approach. Moreover, the approach can be easily automated via an electronic circuit. This approach is also very inexpensive as it requires simple hardware and consumes no reagents.

The invention has direct applications to proteomics research, spanning from the health care industry, medical research, homeland security (bioweapons detection). It can be applied to techniques to identify proteins, mixtures of proteins, or the source of proteins as in the identification of microorganisms.

The advantages of this methodology are its fast speed, simplicity and low cost of the device, amino acid site-specificity, low chemical noise, and easy interfacing to MS instrumentation.

### BRIEF DESCRIPTION OF THE DRAWINGS

FIGS. 1 are graphical representations of the effect of pyrolysis temperature on the fragmentation of the peptide Angiotensin II; product resulting from C-terminus cleavage at aspartic acid is observed at m/z 931.6.

FIG. 2 is the tandem mass spectrum of Angiotensin II pyrolysis product at m/z 931.6.

FIG. 3 are graphs of the ESI-mass spectrum of pyrolysis products of the VIP (1-12) peptide showing site-specific cleavage at the two aspartic acid sites (top spectrum) and the ESI-mass spectrum of pyrolysis products of the VSV-G peptide (bottom spectrum).

FIG. 4 are graphs of the tandem mass spectra of pyrolysis products of the VIP (1-12) peptide, confirming their sequences.

FIG. 5 are graphs of the MALDI-mass spectrum of pyrolysis products of the protein lysozyme (14 kDa), indicating the peptide product detected (top spectrum) and the ESI-tandem mass spectrum of the precursor ion at m/z 1201.6, confirming that sequence information is preserved after protein pyrolysis.

FIG. 6(a) is a diagrammatical view of the on-probe pyrolyzer interfaced to the DESI source; FIG. 6(b) is a diagrammatical view of the on-probe pyrolyzer.

FIG. 7(a) is a diagrammatical view of the on-probe pyrolysis (220° C., 11 s) DESI-mass spectrum of Angiotensin II (inset: before pyrolysis DESI-mass spectrum); site-specific cleavage is induced at the C-terminus of aspartic acid. Ions at m/z 1028 and 1011 are the result of dehydration and deamination reactions, and FIG. 7(b) is a diagrammatical view of the on -probe pyrolysis DESI-tandem mass spectrum of the ion at m/z 931.

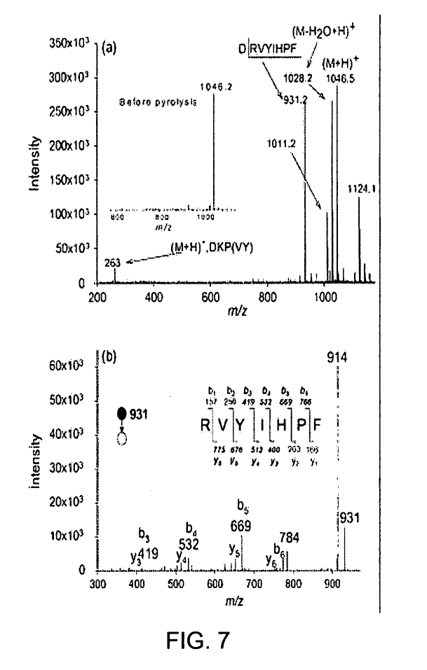

FIGS. 8(a-c) are diagrammatical views of (a) the on -probe pyrolysis DESI-mass spectrum of the VIP peptide showing site-specific cleavages at the two aspartic acids amino acids (inset: before pyrolysis DESI-mass spectrum); and the on-probe pyrolysis DESI-tandem mass spectrum of pyrolytic product at (b) m/z 1086 and (c) m/z 553.

FIG. 9(a) is a diagrammatical view of the on-probe pyrolysis DESI-mass spectrum of lysozyme (inset: before pyrolysis DESI-mass spectrum); and FIG. 9(b) a diagrammatical view of the on-probe pyrolysis DESI-tandem mass spectrum of the ion at m/z 1201.

FIG. 10 is the on-probe pyrolysis DESI-mass spectrum of the protein RNase A (inset: before pyrolysis DESI-mass spectrum).

FIG. 11(a) is the DESI-mass spectrum of PEG 2000 before pyrolysis, and FIG. 11(b) is the on-probe pyrolysis (250° C., 30 min) DESI-mass spectrum of PEG 2000 (inset: zoomed mass spectrum in the range 840-970 Da).

### DETAILED DESCRIPTION OF PREFERRED EMBODIMENTS

### EXAMPLE 1

### Pyrolysis-Induced Cleavage at Aspartic Acid Residue in Peptides and Proteins

Chemicals. Peptides used were: (A) Angiotensin II, human, DRVYIHPF; (B) VIP (1-12) peptide, HSDAVFTDNYTR; and (C) VSV-G peptide, YTDIEMNRLGK (all from AnaSpec, San Jose, Calif.). Lysozyme protein (from Sigma-Aldrich, St. Louis, Mo.) was used without further purification. All solvents used for sample preparation and MS measurements were HPLC grade (Burdick & Jackson, Muskegon, Mich.), and the formic acid (96%) was ACS Reagent grade (Aldrich, St. Louis, Mo.).

Pyrolyzer Design and Pyrolysis Procedure. Approximately a 1 mg solid sample of peptide or protein was pyrolyzed under ambient conditions. Samples were placed in a glass tube (length 31 mm and internal diameter 4 mm; Agilent, Santa Clara, Calif., Part # 5180-0841) and heated using a resistance heating wire (Omega, Stamford, Conn., nickel-chromium wire, part # N160-015-50, length 20 cm) enwound around the tube, powered by 13 V alternating current (AC). Temperature was measured in situ using a thermocouple probe (model HH12A, Omega Company, Stamford, Conn.) reaching down the bottom of the glass tube. The sample was heated for 10 s under atmospheric condition to a final temperature of 220° C. The nonvolatile pyrolysis residue was collected by washing/extracting the inside of the tube with 1 mL of a 50/50 (v/v) methanol-water solution with 0.1% formic acid (FA).

Mass Spectrometry. The extracted solution of pyrolysis products was directly analyzed using a quadrupole ion-trap MS (LCQ classic, Finnigan, San Jose, Calif.) equipped with a nano-Electrospray Ionization (nano-ESI) source by infusing it into the mass spectrometer at a flow rate of 3 μL/min via a 250-μL syringe. Tandem MS (MS/MS) was conducted with the following parameters: activation q of 0.250; isolation width was 1 amu, and the percentage relative collision energy was in the range of 25-40% and was adjusted such that the relative abundance of the precursor ion in the product ion spectrum was approximately 30-50% relative intensity.

MALDI-MS experiments were performed using a MALDITime-of-Flight MS (Voyager DE-PRO, Applied Biosystems, Foster City, Calif.) instrument equipped with a N₂ laser and operated in the reflectron mode. The matrix R-cyano-4-hydroxy-cinnamic acid (CHCA) (Aldrich) was used for all measurements and was prepared by dissolving 10 mg of CHCA in a 1 mL solution of 1:1 acetonitrile/water with 0.1% trifluoroacetic acid (TFA) (Pierce Chemical Co., Rockford, Ill.). The extracted solution of pyrolysis products was directly mixed with the matrix at different volume ratios and air-dried onto a MALDI plate.

### Results and Discussion

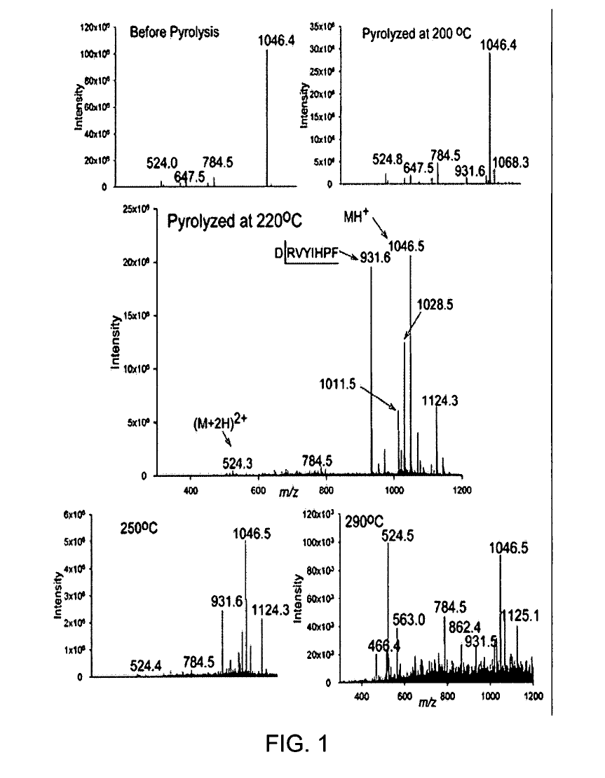

Analysis of Nonvolatile Pyrolysis Products of Peptides. Three peptides containing aspartic acid were pyrolyzed, their nonvolatile products were analyzed by ESI-MS, and their amino acid sequences were confirmed by tandem MS: (A) Angiotensin II, human, DRVYIHPF; (B) VIP (1-12) peptide, HSDAVFTDNYTR; and (C) VSV-G peptide, YTDIEMNRLGK. FIG. 1 shows the full scan mass spectra of peptide A before and after pyrolysis at different temperatures.

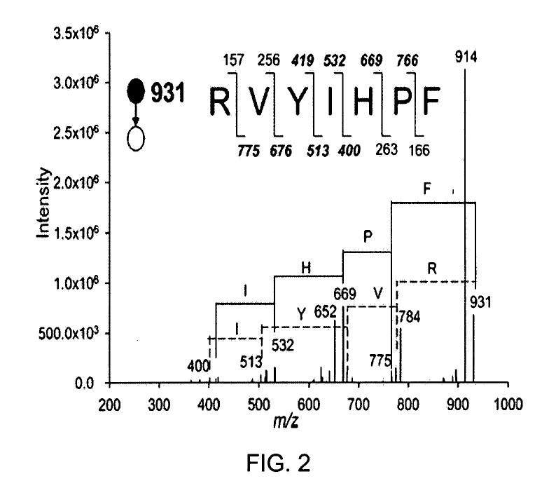

For the peptides tested at pyrolysis temperatures of 200° C. and lower (data not shown), no significant pyrolysis fragments were detected. On the other extreme, at a pyrolysis temperatures of 290° C. and higher (data not shown), extensive fragmentation products were observed, most likely due to peptide carbonization. At pyrolysis temperatures between 220 and 250° C., the pyrolysis fragment for peptide A due to C-terminal cleavage at aspartic acid was detected at m/z 931.6. Tandem MS (MS/MS) of this ion (FIG. 2) confirmed the sequence RVYIHPF, the product of a C-terminus cleavage at the aspartic acid residue of peptide A. Other peaks present in the spectrum resulted from consecutive loss of water, observed at m/z 1028.5 (C-terminus oxazolone formation) (Zhang, S.; Basile, F. Investigation of Non-Volatile Pyrolysis Products of Proteins Using Electrospray Ionization Multi-step Tandem Mass Spectrometry. 54th ASMS Conference, 2006), and loss of ammonia, observed at m/z 1011.5 (from arginine).

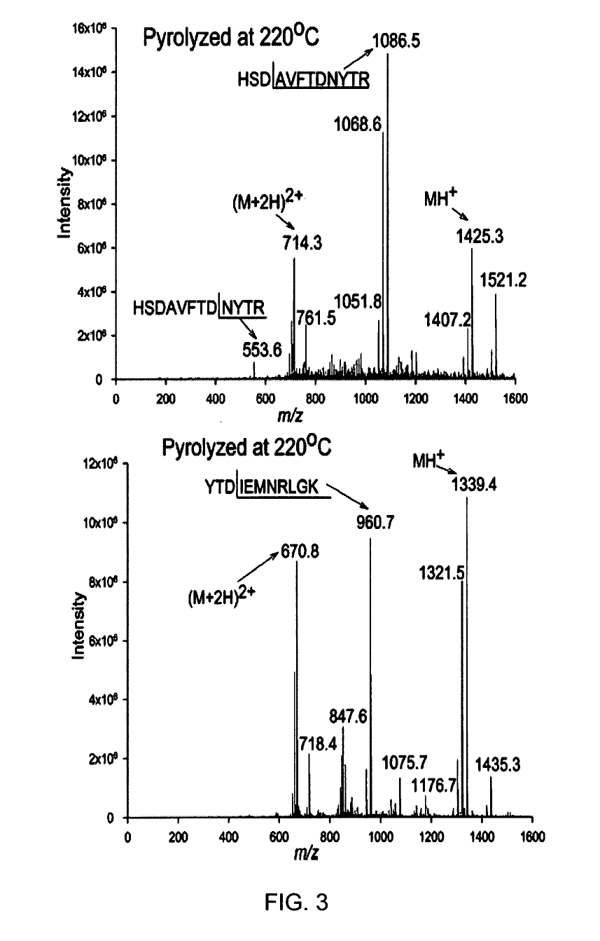

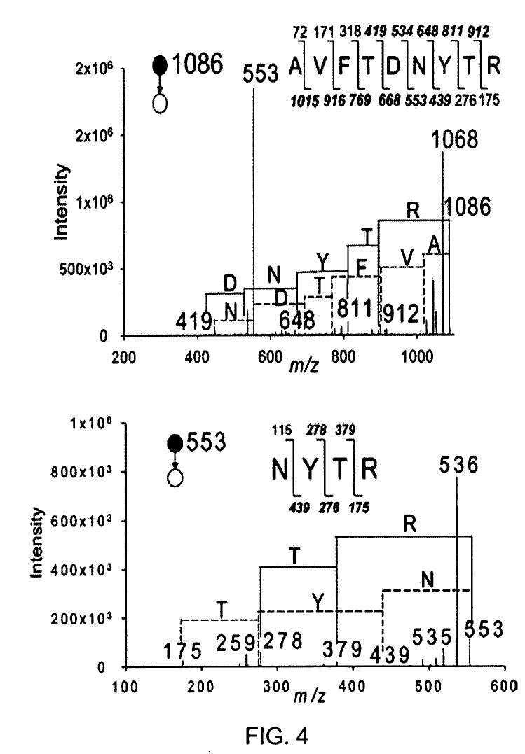

Site-specific pyrolysis-induced cleavage was also observed for peptide B, which contains two aspartic acid residues (ESI-mass spectrum shown in FIG. 3, top). After pyrolysis at 220° C., two nonvolatile peptide products were observed at m/z 1086.5 (AVFTDNYTR) and m/z 553.6 (NYTR), corresponding to cleavages at each of the two aspartic acid C-terminus sites. Amino acid sequences of these pyrolysis products were confirmed by MS/MS measurements (shown in FIG. 4). Also, possible peptide oxidation products were observed at m/z 1521 for peptide B and at m/z 1435 for peptide C, and their structures are currently being investigated. Similar results were observed for peptide C (FIG. 3, bottom).

These results demonstrate that pyrolysis at temperatures between 220 and 250° C. favors cleavage in peptides at the C-terminus of aspartic acid. Peptide fragmentation at the C-terminus of aspartic acid is believed to proceed via the formation of a five-member cyclic anhydride followed by hydrolysis, because pyrolysis is performed in air and at atmospheric pressure (Scheme 1) (Inglis, S. A. Cleavage at aspartic acid. Methods Enzymol. 1983, 91, 324-332).

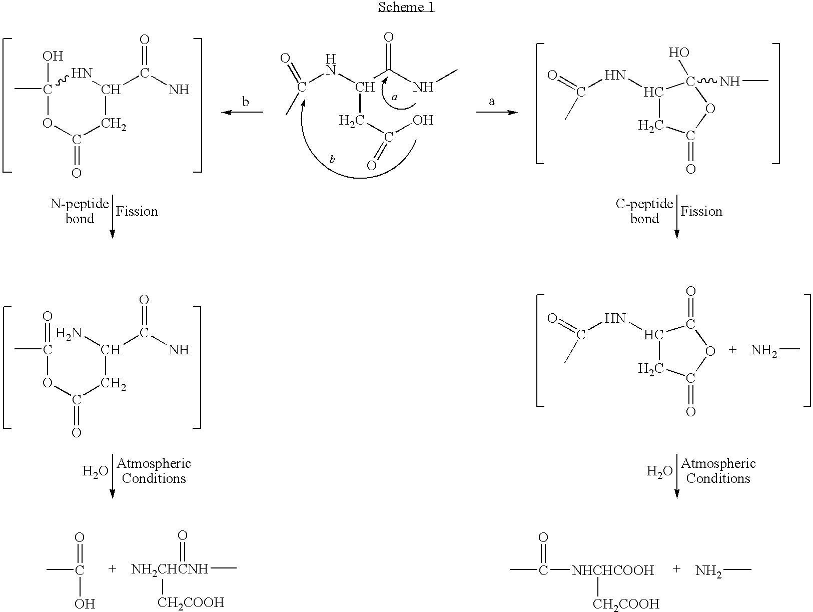

The overall susceptibility of the aspartic acid group to internal cleavage may stem from the fact that the \hat{α}-carboxyl group (i.e., side-chain carboxyl group) acts as a proton donor and its hydroxyl oxygen as a nucleophile toward the adjacent carbonyl carbons in the peptide bond. Reaction path “a” in Scheme 1 leads to the formation of a five-member ring, while path “b” forms a six-member ring species. Hydrolysis of these cyclic intermediates results in C- or N-terminus cleavages of the aspartic acid residue, respectively. The six-member ring molecule leading to the N-terminus cleavage is expected to be thermodynamically more stable than the five-member cyclic anhydride molecule (Loudon, G. M. Organic Chemistry; Addison-Wesley Publishing Co.: Massachusetts, 1983). However, only C-terminus cleavage products have been detected under pyrolysis conditions, and these would result from the formation of the five-member ring species. Hence, it is hypothesized that the reaction path “a” leading to the pyrolysis-induced C-terminus cleavage is kinetically favored rather than thermodynamically controlled.

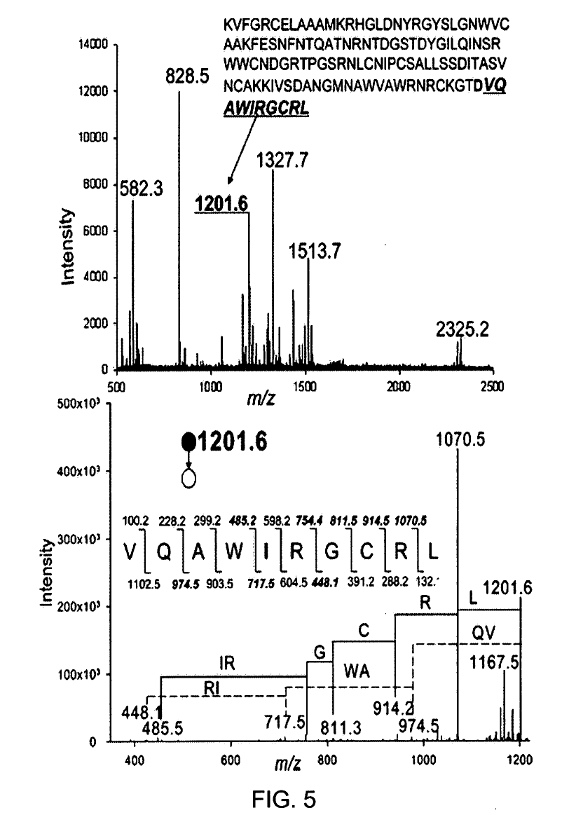

Analysis of Nonvolatile Pyrolysis Products of Lysozyme. The potential of this methodology to digest intact proteins to smaller peptides for subsequent MS/MS analyses was further tested. MALDI-MS analysis of the nonvolatile pyrolysis products of the protein lysozyme resulted in a series of strong signals in the mass range of 500-2500 u, indicating that complete degradation or carbonization of the protein does not occur at 220° C. (FIG. 5, top). Moreover, the ion at m/z 1201.6 observed in the MALDI-mass spectrum matches one of the expected products corresponding to the cleavage at the C-terminus of aspartic acid in lysozyme, the peptide (D) VQAWRGCRL.

Analysis of this pyrolysis digestion product by ESI-MS/MS (MS/MS of m/z 1201.6) yielded sequence information confirming the peptide amino acid sequence (FIG. 5, bottom). Moreover, fragment ion data were used for successful protein identification via database search (Mascot score 52; threshold score for significant homology was 43; Matrix Science, UK). Hence, the observed pyrolysis digestion product at m/z 1201.6 corresponds to the C-terminal peptide in the lysozyme protein sequence, confirming that the pyrolysis product is derived from the protein and that sequence information is conserved. We are currently investigating factors affecting sequence coverage and the structure of additional pyrolysis products observed (e.g., dehydration, deamination, and oxidation products), the effect of neighboring amino acids on cleavage, and the ability of the method to cleave at other aspartic acid residues, that is, other than those near the C-terminal of the protein sequence.

Conclusion. The ability of pyrolysis-based digestion methods to produce sequence-specific biomarkers has been demonstrated for peptides and the protein lysozyme. This approach offers the possibility of developing rapid and field-portable proteomic-based methods to detect and identify biological samples, for example, protein toxins and/or pathogenic bacteria (e.g., Bacillus anthracis). In this particular application, protein sequence coverage is not a requirement, but, rather, the reproducibility and simplicity of the pyrolysis method is used to generate biological-specific biomarkers.

### EXAMPLE 2

### Pyrolysis Device and Procedures

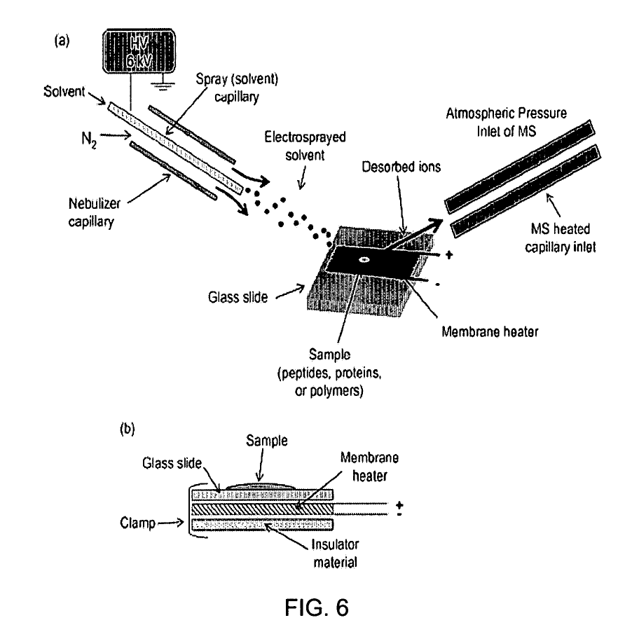

A diagram of the on-probe pyrolyzer interfaced to the DESI source is shown in FIG. 6. A homebuilt DESI source was interfaced with a quadrupole ion trap MS (LCQ Classic, Thermo Electron, San Jose, Calif.) and was operated in the positive ion mode. The on-probe pyrolyzer consisted of a membrane heater (Model #HM6815, Minco, Minneapolis, Minn.) placed underneath a removable glass slide held tightly together with a clamp (FIG. 6b). The sample to be pyrolyzed was placed directly on the center of the glass slide. The membrane heater was powered by alternating current (AC) from a transformer (Model #3PN116C, Superior Electric, Farmington, Conn.) and heating and final pyrolysis temperature were controlled by adjusting the voltage of the transformer and the heating time. For our current setup, a voltage of 20 V applied for 11 s resulted in a final pyrolysis temperature of 220° C. These values for pyrolysis temperature and time were used for all biological samples analyzed in this study. The glass slide surface temperature was measured in situ using a thermocouple probe (Model #HH12A, Omega Company, Stamford, Conn.) placed in direct contact. After sample pyrolysis, the probe was cooled to room temperature (<5 min) and the DESI-MS analysis carried out. This setup is amenable to conducting pyrolysis in either the off-line or on-line mode with the DESI source, that is, a sample placed on a slide can be pyrolyzed in a furnace under controlled atmospheric conditions and later analyzed by DESI-MS. However, all measurements in this report were performed in the on-line configuration (FIG. 6a).

Several model samples were tested with this new on-probe pyrolyzer DESI-MS instrument. Peptides analyzed included Angiotensin 11-human, of sequence DRVYIHPF, and the peptide VIP (1-12), of sequence HSDAVFTDNYTR (both from AnaSpec, San Jose, Calif.). The proteins used were lysozyme and RNase A, and the synthetic polymer used was poly(ethylene glycol) (PEG 2000) (all from Sigma-Aldrich, St. Louis, Mo.). Methanol, water (from Burdick & Jackson, Muskegon) and tetrahydrofuran (THF, from EMD Chemicals, San Diego, Calif.) were used for sample preparation and MS measurements (all HPLC grade). About a 1 mg sample of the peptides was dissolved in 200 μL of methanol, and the entire solution air-dried on a glass slide (covering a surface area approx. 6 cm², ˜0.1 mg sample/cm²) and placed on the on-probe pyrolyzer. Lysozyme and RNase A were prepared in a similar fashion, but dissolved in water. For poly(ethylene glycol), about 10 mg of PEG 2000 was dissolved in 1 mL of THF, air-dried on a glass slide (˜1 mg/cm²), placed on the on-probe pyrolyzer and heated to a final temperature of 250° C. for 30 min.

### DESI and Mass Spectrometry Parameters

The DESI source was operated with a high voltage of 6 kV applied to the spraying solvent. The spraying solvent consisting of 50% methanol in water (v/v) was delivered at a flow rate of 7 μL/min via a syringe pump. All mass spectra were collected in spectral average mode. The pressure of the DESI nebulizer gas (N₂) was set as 250 psi.

Tandem MS (MS/MS) measurements were conducted with the following parameters: activation q of 0.250; isolation width was 1 amu and the percentage relative collision energy was in the range of 25-40%, and was adjusted to get a precursor ion peak of 25% relative intensity or less (when possible).

### Results and Discussion

The utility and versatility of the DESI source interfaced with the on-probe pyrolyzer for the analysis of non-volatile pyrolysis products were demonstrated with several model compounds that included peptides, proteins and a synthetic polymer.

### EXAMPLE 3

### On-probe Pyrolysis DESI-MS Analysis of Biomolecules

As described in Example 1, the site-specific pyrolysis-induced cleavage at the amino acid aspartic acid (letter symbol “D”) in both peptides and proteins has been achieved by heating samples to a temperature of 220-250° C. for 10 s under atmospheric pressure conditions. Peptides and proteins in this previous study were pyrolyzed in an open-ended tube furnace, extracted with a suitable solvent and analyzed by ESI-MS and MS/MS to characterize and identify non-volatile pyrolysis cleavage products. In this Example, the same samples were pyrolyzed on-probe and products were analyzed in situ by DESI-MS, bypassing the sample extraction, transfer, and ESIinfusion steps. In the ESI-MS study and the DESI-MS study here described, pyrolysis of peptides and proteins above 300° C. produced complete charring of the polypeptide backbone.

Pyrolysis induced site-specific cleavage at aspartic acid has was observed mostly at low temperature pyrolysis. However, this pyrolysis cleavage reaction is not exclusive in biomolecules as other pyrolysis fragments have been detected and the system here described is presently being used to further characterize the structure and nature of these pyrolysis fragments.

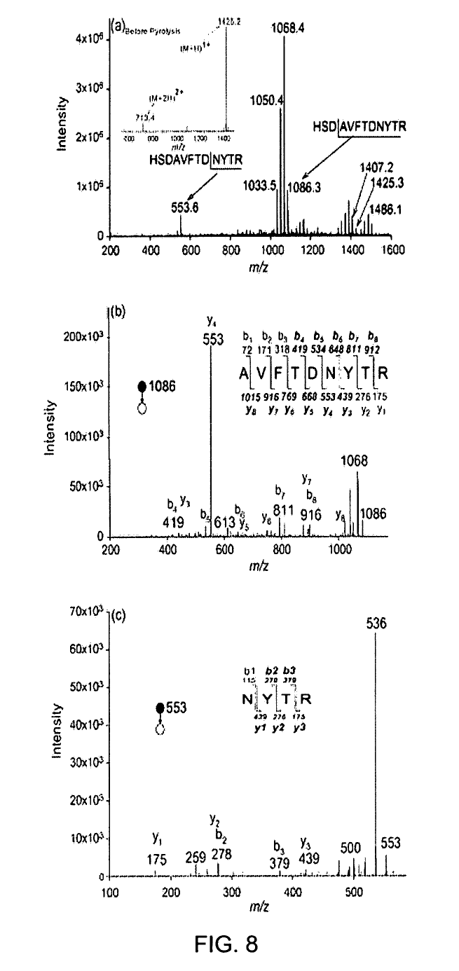

FIG. 8 illustrates the DESI-mass spectra before and after onprobe pyrolysis of the peptide Angiotensin II, along with the tandem mass spectrum of the pyrolytic product at m/z 931. The DESI-mass spectrum of the non-volatile products also shows the formation of a dehydration product at m/z 1028.2, a possible oxidation product at m/z 1124.1 (of yet unknown structure) and the product of the pyrolysis induced site-specific cleavage at aspartic acid at m/z 931.2 (the D-cleavage pyrolysis peptide product). Tandem MS data of the ion at m/z 931 confirms that sequence-specific information is preserved after low temperature pyrolysis of peptides.

The above measurement demonstrates the simplicity and speed of analysis of pyrolysis residues with the on-probe pyrolyzer coupled to a DESI-MS system. No solvents were required for residue extraction and solubilization, assuring the analysis of the entire pyrolysis product mixture (i.e., the nonvolatile fraction, vide infra). However, it is reassuring to note that all products detected in the on-probe pyrolysis DESI-MS analysis in FIG. 8 were also observed in the open-ended tube furnace pyrolysis and ESI-MS analysis, which required sample extraction and solubilization. It is important to note that lower MW products like diketopiperazines (DKP) known to be generated under Curie-point and atmospheric pyrolytic conditions were only observed in the analysis of the Angiotensin II peptide (signal at m/z 263 corresponding to the (M+H)+DKP of VY). This may be due to several factors: first, volatile DKP products may have been lost during the pyrolysis process since the on-probe pyrolyzer is operated at atmospheric pressure. Second, early work on the formation of DKP from dipeptides (D. Gross, G. Grodsky, J. Am. Chem. Soc. 77 (1955) 1678-1680; H. J. Svec, G. A. Junk, J. Am. Chem. Soc. 86 (1964) 2278-2282) found that only a small percentage (˜7%) of the original dipeptide was converted to DKP at 215° C. And finally, ionization suppression of the DKP (M+H)+signals within the desorbed DESI droplets may take place, especially if analyzing a complex mixture of pyrolytic products with dissimilar droplet surface activities or DKPs in mixtures with peptides containing highly basic groups (i.e., arginine), as it is the case here.

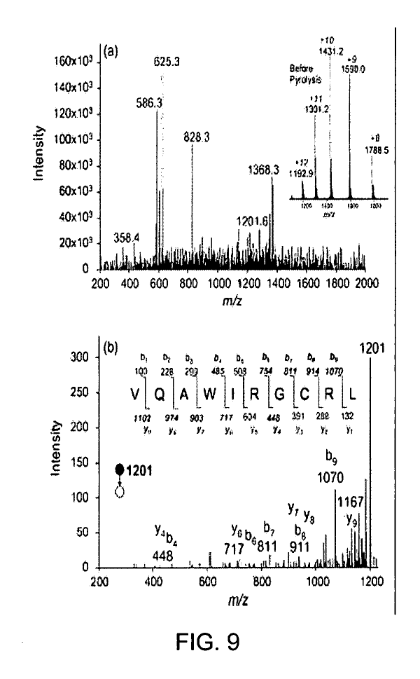

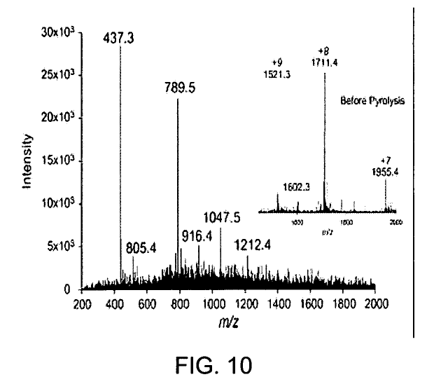

FIG. 9 illustrates the on-probe pyrolysis and DESI-MS analysis of another peptide, VIP (1-12) peptide, which contains two aspartic acid residues. Specifically, the on-probe pyrolysis DESI-mass spectrum (FIG. 9a) is characterized by the ions at m/z 553.6 and 1086.3, which correspond to the expected products due to site-specific cleavages at the two aspartic acid residues (D-cleavage pyrolysis). This D-cleavage pyrolysis is believed to proceed via a similar mechanism as in the solution phase reaction, that is, the formation of a five-member cyclic anhydride followed by hydrolysis. Similar results were also obtained in the open-ended tube furnace (at atmospheric pressure conditions) and solvent extraction ESI-MS analysis of the pyrolysis residues. Other ions observed at m/z's 1068 and 1050 result from consecutive losses of water and ammonia (from arginine) from the pyrolysis fragment at m/z 1086.3, and these ions were also observed in the off-line pyrolysis and extraction ESI-MS measurements. FIG. 9b and c show the on-probe DESI-tandem mass spectra of the pyrolysis products at m/z 553 and 1086, confirming their sequences and the site-specificity of the pyrolysis cleavage at aspartic acid. Also, the on-probe pyrolysis DESI-MS instrument was used to analyze the non-volatile pyrolysis products of the proteins lysozyme (MW 14.3 kDa) and RNase A (MW 13.7 kDa). FIG. 10 shows the DESI-mass spectra of lysozyme before and after pyrolysis and the DESI-tandem mass spectrum for the ion at m/z 1201. This ion corresponds to the protein C-terminus peptide due to D-cleavage pyrolysis as confirmed by the DESI-tandem mass spectral data in FIG. 9b. In previous work, it was successfully shown that this sequence information can be used to identify the protein via a proteomic-based approach and database search (e.g., MASCOT, Matrix Science Ltd., London, UK). FIG. 10 illustrates the on-probe pyrolysis DESI-MS analysis of the protein RNase A with the detection of several prominent pyrolysis products observed at m/z's 437.3, 789.5, 916.4, 1047.5 and 1212.4; however, none of the main signals observed match expected products resulting from D-cleavage pyrolysis. In previous investigations and in this study, the D-cleavage pyrolysis peptide product was derived from the C-terminus of the protein sequence, and not from cleavages of internal D groups.

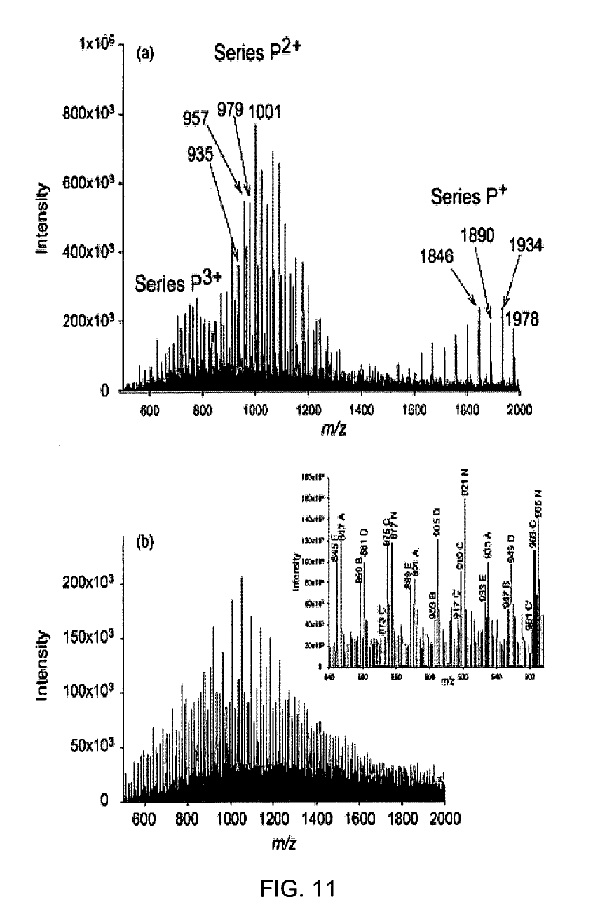

On-probe DESI-MS analysis of poly(ethylene glycol) Poly(ethylene glycol) with an average molecular weight of 2000 g/mol (PEG 2000) was used to test the ability of the on-probe pyrolyzer DESI-MS instrument to study thermal degradation processes in synthetic polymers. FIG. 11 shows the DESI-mass spectra of the PEG 2000 before and after onprobe pyrolysis at 250° C. for 30 min. The DESI-mass spectrum of untreated PEG 2000 (FIG. 6a) shows a distribution of singly charged ions near m/z 2000 as their (M+Na)⁺ ions (monomer unit Dm=44 u) denoted in the spectrum as the P⁺-series. A doubly charged P²⁺-series is also observed near m/z 1000 (monomer unit Dm=22 u) and is composed of both (M+2Na)²⁺ and (M+Na+K)²⁺ ions. On the other hand, the on-probe pyrolyzed DESI-mass spectrum of PEG 2000 (FIG. 11b and inset) is strikingly different, with the P⁺ series shifted to an average molecular weight near m/z 1000, while the p²⁺ and p³⁺ were not detected. Careful inspection of this mass spectrum (FIG. 11b inset) reveals the presence of several series of poly(ethylene glycol) with different end groups, and these are labeled using nomenclature coined by Voorhees et al. (Voorhees, K. J., Baugh, S. F., Stevensen, D. N. J. Anal. Appl. Pyrol. 30 (1994) 47-57)). The spectrum in FIG. 11b is dominated by the unmodified hydroxylpoly (ethylene) glycol series (labeled N in the spectrum), methyl ether series (A), aldehyde series (C) and the ethyl ether series (D). Less dominant, but present, are the vinyl ether series (B), the methyl ether/aldehyde series (E) and the methyl-vinyl ether series (C0). These results are in direct agreement with previous MALDI-MS studies (Lattimer, R. P. J. Anal. Appl. Pyrol. 56 (2000) 61-78) of the pyrolyzate residues of poly(ethylene glycol), proving that the on-probe pyrolysis DESI-MS technique described in this report yields comparable results. Moreover, the on-probe pyrolysis DESI-MS approach does not require matrix compounds, decreasing sample preparation time and avoiding matrix-sample adducts that can add to the chemical noise in the mass spectrum. Also, in this Example, no cationizing agent was added to either the polymer sample or the DESI solvent, and we believe the source of the Na⁺ ions to be the glass slide and/or from trace amounts contained in the DESI solvent.

Conclusion An on-probe pyrolyzer interfaced with desorption electrospray ionization (DESI) mass spectrometry and tube pyrolysis with sample extraction were successfully demonstrated to induce site specific cleavage at aspartic acid in biological samples. These results are in agreement with analyses of non-volatile pyrolysis products performed either by ESI-MS or MALDI-MS, which were pyrolyzed off-line and required sample extraction and solubilization. For biological samples and using the on-probe pyrolyzer DESI-MS system, it has here been demonstrated that pyrolysis residues of peptides and the protein lysozyme retain sequence information useful for proteomic-based protein identification. Moreover, these results demonstrate that atmospheric pressure pyrolysis can induce a variety of products that include site-specific cleavages at aspartic acid, dehydration reactions in peptides and proteins, and other products. For the analysis of poly(ethylene glycol), the on-probe pyrolysis DESI-MS system yielded data and information equivalent to previous MALDI-MS analysis, where the use of a matrix compound and cationizing agent were required. Quantitative to semi-quantitative analysis with DESI-MS is feasible, although quantitation of pyrolysis products was not addressed in this work. Overall, results from this work have demonstrated clear advantages of combining an on-probe pyrolyzer with a DESI source that include: minimum sample preparation, no sample extraction or transfer after pyrolysis, atmospheric pressure pyrolysis, rapid and atmospheric pressure detection by DESIMS, the ability for sample archival (samples on slides), and tandem-MS (if using a multistage-MS system).

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

We claim:

1. A method of digesting peptides, comprising heating a peptide sample to between about 180° C. and about 250° C., in a period of between about 5 seconds and about 30 seconds to cleave the peptide at a site-specific location.

2. A method as defined in claim 1, wherein the temperature is at least about 220° C. and the time period is less than about 10 seconds.

3. A method as defined in claim 1, wherein the peptide sample is selected from one or more of the group consisting of a pure protein, a mixture of proteins, whole microorganisms, and intact tissue.

4. A method as defined in claim 1, wherein the method is carried out in the absence of protolytic enzymes.

5. A method as defined in claim 1, wherein the site-specific location is the C-terminus of aspartic acid.

6. A method of analyzing a peptide sample, comprising the steps of:
(a) heating the peptide sample to between about 180° C. and about 250° C., in a period of between about 5 seconds and about 30 seconds to cleave the peptide at a site-specific location;
(b) electrospraying the digested sample with a solvent to produce desorbed ions of components of the digested sample; and
(c) detecting the desorbed ions.

7. A method as described in claim 6, wherein the step of detecting the desorbed ions is by mass spectrometry.

8. A method as described in claim 6, wherein the solvent is free of cationizing agents.

9. A method as described in either claim 1 or claim 6, wherein the method is performed at atmospheric pressure.

10. Apparatus for analyzing a peptide sample, comprising:
(a) a heating element having a surface on which the sample is deposited and which heats the peptide sample to between about 180° C. and about 250° C., in a period of between about 5 seconds and about 30 seconds to cleave the peptide at a site-specific location;
(b) an electrospray device that subjects the digested sample to a solvent spray to produce desorbed ions of components of the digested sample; and
(c) a detector for detecting the desorbed ions.
